(19) United States
(12) Patent Application Publication
(10) Pub. No.: US 2019/0374780 A1
(43) Pub. Date: Dec. 12, 2019

(54) MEDICAL IMPLANTS POWERED BY REVERSE ELECTRODIALYSIS

(71) Applicant: The Board of Trustees of the University of Arkansas, Little Rock, AR (US)

(72) Inventors: Jamie HESTEKIN, Fayetteville, AR (US); Christa HESTEKIN, Fayetteville, AR (US); Chase SMITH, Fayetteville, AR (US); Brigitte RODGERS, Fayetteville, AR (US); Pedro Rafael Lima VIEIRA, Rio Grande Do Norte (BR)

(21) Appl. No.: 16/331,871

(22) PCT Filed: Sep. 11, 2017
(86) PCT No.: PCT/US17/50972

§ 371 Date: Mar. 8, 2019

Related U.S. Application Data
(60) Provisional application No. 62/385,565, filed on Sep. 9, 2016.

(51) Int. Cl. A61N 1/378; H01M 8/22; H01M 16/00

(52) U.S. Cl. CPC A61N 1/3785; A61N 1/3956; H01M 16/006; H01M 8/227

(57) ABSTRACT

In one aspect, medical implant power sources employing reverse electrodialysis principles are described herein. For example, a power source for a medical implant comprises an anode and cathode adjacent to a membrane stack, the membrane stack comprising alternating anion and cation exchange membranes defining diluate and concentrate fluid compartments. The power source includes a first conduit for delivering a diluate blood stream to the one or more diluate fluid compartments and a second conduit for delivering a concentrate blood stream to the one or more concentrate fluid compartments, wherein the concentrate blood stream has an ionic concentration higher than the diluate blood stream.

### RELATED APPLICATION DATA

The present application hereby claims priority pursuant to 35 U.S.C. § 119(e) to U.S. Provisional Patent Application Ser. No. 62/385,565 filed Sep. 9, 2016 which is incorporated herein by reference in its entirety.

### STATEMENT OF GOVERNMENT RIGHTS

This invention was made with government support under REU Grant No. EEC-1359306 awarded by the National Science Foundation. The government has certain rights in the invention.

### FIELD

The present invention relates to medical devices and, in particular, to medical implants powered by reverse electrodialysis systems.

### BACKGROUND

Implantable medical devices constitute an industry having valuation in the tens of billions of dollars. Numerous medical implants require power sources for proper function. Such power sources are generally limited to battery architectures of small footprint. Battery architectures can vary depending on the power requirements of the medical implant. Several current architectures are based on lithium ion systems, including lithium/iodine batteries, lithium/manganese dioxide batteries, lithium/carbon monofluoride batteries and lithium/silver vanadium oxide batteries. While offering several advantages, current battery architectures suffer significant disadvantages of a finite lifetimes, hazardous materials and high replacement costs, thereby calling for the development of new power sources for medical implants.

### SUMMARY

In one aspect, medical implant power sources employing reverse electrodialysis principles are described herein. Briefly, a power source for a medical implant comprises an anode and cathode adjacent to a membrane stack, the membrane stack comprising at least one ion exchange membrane defining a diluate compartment and concentrate compartment. The ion exchange membrane can be a cation exchange membrane or anion exchange membrane. In some embodiments, the membrane stack comprises alternating anion and cation exchange membranes defining one or more diluate and concentrate fluid compartments. The power source includes a first conduit for delivering a diluate blood stream to the one or more diluate fluid compartments and a second conduit for delivering a concentrate blood stream to the one or more concentrate fluid compartments, wherein the concentrate blood stream has an ionic concentration higher than the diluate blood stream.

In another aspect, methods of powering a medical implant are described herein. In some embodiments, a method of powering a medical implant comprises providing a power source comprising an anode and cathode adjacent to a membrane stack, the membrane stack comprising at least one ion exchange membrane defining a diluate compartment and concentrate compartment. In some embodiments, the membrane stack comprises alternating anion and cation exchange membranes defining one or more diluate and concentrate fluid compartments. A diluate blood stream is flowed into the one or more diluate compartments via a first conduit and a concentrate blood stream is flowed into the one or more concentrate compartments via a second conduit, wherein the concentrate blood stream has an ionic concentration higher than the diluate blood stream. Ions are passed through the ion exchange membrane from the concentrate blood stream, wherein the power source is connected to the medical implant for extraction of electrical current from the power source. In embodiments employing anion and cation exchange membranes, anions are passed from the concentrate blood stream through the anion exchange membrane and cations are passed from the concentrate blood stream through the cation exchange membrane, wherein the power source is connected to the medical implant for extraction of electrical current from the power source.

In a further aspect, medical devices are described herein. A medical device, in some embodiments, comprises an implant and a power source coupled to the implant. The power source comprises an anode and cathode adjacent to a membrane stack, the membrane stack comprising at least one ion exchange membrane defining a diluate compartment and concentrate compartment. In some embodiments, the membrane stack comprises alternating anion and cation exchange membranes defining one or more diluate and concentrate fluid compartments. The power source includes a first conduit for delivering a diluate blood stream to the one or more diluate fluid compartments and a second conduit for delivering a concentrate blood stream to the one or more concentrate fluid compartments, wherein the concentrate blood stream has an ionic concentration higher than the diluate blood stream.

These and other embodiments are described further in the following detailed description.

### BRIEF DESCRIPTION OF THE DRAWINGS

FIG. 1 illustrates a membrane stack and associated electrodes of a power source according to one embodiment described herein.

FIG. 2 illustrates functioning of the membrane stack according to one embodiment described herein.

FIG. 3 illustrates a power source according to one embodiment described herein.

FIG. 4 and FIG. 5 illustrate several results of power generation simulations based on sodium chloride solutions mimicking diluate and concentrate blood streams.

FIG. 6 and FIG. 7 provide power density over time comparisons between a reverse electrodialysis power source described herein and a biobattery architecture based on the enzymatic breakdown of glucose.

FIG. 8 illustrates a linear increase in power density with increasing number of cells of a power source described herein.

FIG. 9 illustrates a linear increase in voltage with increasing number of cells of a power source described herein.

FIG. 10 illustrates a linear dependence of powder density relative to the magnitude of the sodium gradient between the diluate and concentrate blood streams for a power source described herein.

FIG. 11 illustrates a membrane stack and associated electrodes of a power source according to one embodiment described herein.

### DETAILED DESCRIPTION

Embodiments described herein can be understood more readily by reference to the following detailed description, examples and drawings. Elements, apparatus, and methods described herein, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

As described herein, a power source for a medical implant comprises an anode and cathode adjacent to a membrane stack, the membrane stack comprising at least one ion exchange membrane defining a diluate compartment and concentrate compartment. The ion exchange membrane can be a cation exchange membrane or anion exchange membrane. In some embodiments, the membrane stack comprises alternating anion and cation exchange membranes defining one or more diluate and concentrate fluid compartments. The power source includes a first conduit for delivering a diluate blood stream to the one or more diluate fluid compartments and a second conduit for delivering a concentrate blood stream to the one or more concentrate fluid compartments, wherein the concentrate blood stream has an ionic concentration higher than the diluate blood stream.

Turning now to specific components, the anode and cathode of the power source can be constructed of any material and have any dimensions not inconsistent with the objectives of the present invention. For example, the cathode and/or anode can be formed of a material selected from the group consisting of aluminum, titanium, platinum on titanium, iridium on titanium, stainless steel, iron, zinc, nickel, copper, other metals and alloys thereof.

The membrane stack of the power source comprises at least one ion exchange membrane defining a diluate compartment and concentrate compartment. In some embodiments, the membrane stack comprises alternating anion and cation exchange membranes defining diluate and concentrate ionic solution compartments. In some embodiments, the alternating anion and cation exchange membranes define a single diluate compartment and a single concentrate compartment. Moreover, the alternating anion and cation exchange membranes can define a plurality of diluate compartments and a plurality of concentrate compartments. The membrane stack can have any desired number of anion and/or cation exchange membranes not inconsistent with the objectives of the present invention. The number of anion and/or cation exchange membranes can be selected according to several considerations including the desired number cells in the membrane stack. In some embodiments, the membrane stack is limited to a single cell. In other embodiments, the membrane stack comprises a plurality of cells. Cell number of the membrane stack can be selected according to the power requirements of the medical implant and any size constraints imposed by the site at which the power source is implanted in the patient.

Any anion and cation exchange membranes not inconsistent with the objectives of the present invention can be employed. Anion exchange membranes for use in power sources described herein include membranes under the FUMASEP® trade designation, such as FUMASEP® FAS and FAB. Suitable anion exchange membranes also include Tokuyama NEOSEPTA® membranes such as Tokuyama AMX, AMH and ACM. Anion exchange membranes are also commercially available from Ameridia. Typical properties of anion exchange membranes employed in power sources described herein are provided in Table I.

TABLE I
Anion Exchange Membrane Properties

Ion Exchange Capacity 0.6-2.0 meq/g
Selectivity > 90%
Ohmic Resistance < 10 Ω-cm
Thickness 0.5-5 mm

Cation exchange membranes for use in power sources described herein also include membranes under the FUMASEP® trade designation, such as FUMASEP® FKS and FKE. Suitable cation exchange membranes can be obtained from Tokuyama such as Tokuyama CMX, CMS and CMB. Typical properties of cation exchange membranes employed are provided in Table II.

TABLE II
Cation Exchange Membrane Properties

Ion Exchange Capacity 0.6-2.0 meq/g
Selectivity > 90%
Ohmic Resistance < 10 Ω-cm
Thickness 0.5-5 mm

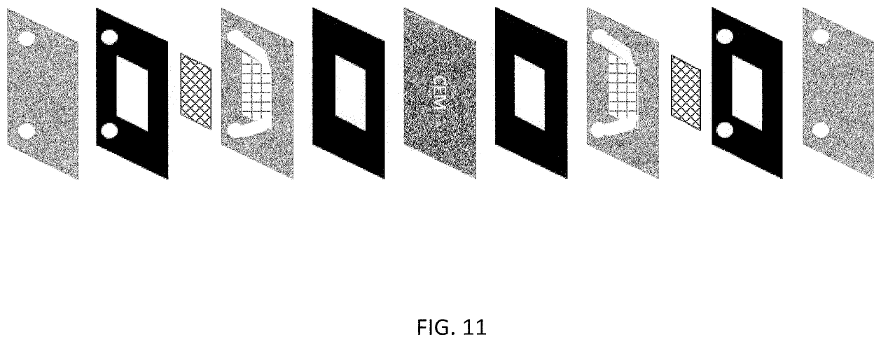

In some embodiments, a cation or anion exchange defines diluate and concentrate compartments for receiving diluate and concentrate blood streams respectively. As described further herein, FIG. 11 illustrates an embodiment wherein a cation exchange membrane (CEM) defines diluate and concentrate compartments. In other embodiments, anion and cation exchange membranes define diluate and concentrate compartments. The diluate and concentrate compartments can have any dimensions not inconsistent with the objectives of the present invention. Compartment dimensions can be selected according to several considerations including diluate and concentrate blood stream flow rates into the compartments. A first conduit delivers the diluate blood stream into the one or more diluate fluid compartments, and a second conduit delivers the concentrate blood stream into the one or more concentrate fluid compartments. The terms diluate blood stream and concentrate blood stream are used relative to one another. For example, the concentrate blood stream exhibits ionic concentration higher than the diluate blood stream. In some embodiments, ionic concentration of the diluate and concentrate blood streams is sodium ion concentration. The sodium ion concentration of the concentrated blood stream can be at least 10 percent higher than the sodium ion concentration of the diluate blood stream. In some embodiments, the sodium ion concentration of the concentrated blood stream is 10-30 percent higher than the sodium ion concentration of the diluate blood stream.

To establish an ionic concentration gradient for the membrane stack, the diluate and concentrate blood streams can be sourced from differing parts of the patient's body. In some embodiments, the diluate and concentrate blood streams are sourced from differing blood vessels. For example, the diluate and concentrate blood streams can be sourced from different veins transporting blood of differing ionic composition. In such embodiments, the power source further comprises a diluate blood stream return conduit and a concentrate blood stream return conduit for returning the blood streams to the proper blood vessels.

In other embodiments, one or more salt cartridges can be employed to establish the ionic concentration gradient for the membrane stack. In such embodiments, blood is split into two streams prior to interfacing with the membrane stack. The first blood stream contacts the salt cartridge thereby increasing the ionic concentration in the first blood stream. The second blood stream does not contact the salt cartridge. By having a higher ionic concentration, the first blood stream is the concentrate blood stream, and the second blood stream is the diluate blood stream.

The salt cartridge can comprise any salt or mixture of salts not inconsistent with the objectives of the present invention. For example, the salt cartridge can employ salts naturally found in the human or animal body including, but not limited to, sodium chloride, potassium chloride and calcium chloride.

The salt cartridge can provide any desired amount of salt to the first blood stream. Amounts of salt imparted to the blood stream can be selected according to several considerations including desired strength of the ionic gradient between the concentrate and diluate blood streams and physiological factors and constraints imposed by the human or animal body. The cartridge, for example, can impart less than 5 mg/ml of salt to the first blood stream per day. In some embodiments, the cartridge imparts 0.5-5 mg/ml of salt to the first blood stream per day. The salt cartridge can have any structural configuration for interacting with a blood stream. In some embodiments, the salt cartridge comprises a reservoir containing a salt solution. A perforated tube carrying the first blood stream passes through the reservoir, wherein salt solution is picked up by the first blood stream. Flow rates of the first blood stream can be varied to impart desired amounts of salt to the blood stream. Perforation size, number and/or density can also be varied to control amounts of salt imparted to the blood stream. Use of a salt cartridge permits the power source to be placed anywhere in the patient's body since concentrate and diluate blood streams are not required to be sourced from specific areas of the patient.

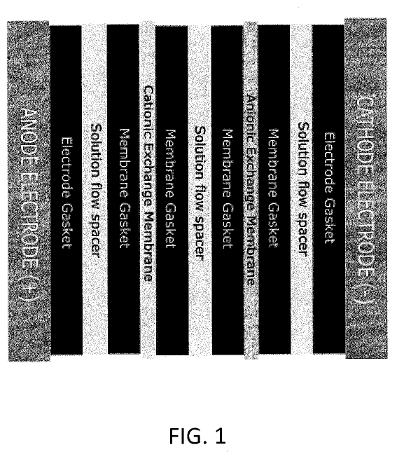

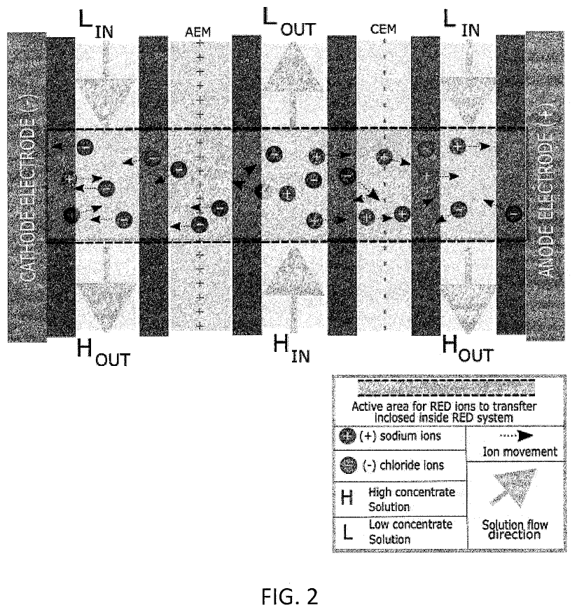

FIG. 1 illustrates a membrane stack and associated electrodes of a power source according to one embodiment described herein. Moreover, FIG. 2 illustrates functioning of the membrane stack of FIG. 1. As illustrated in FIGS. 1 and 2, the membrane stack comprises anion and cation exchange membranes defining a concentrate compartment and diluate compartments. Alternatively, a single ion exchange membrane can be employed to define concentrate and diluate compartments, as illustrated in FIG. 11. In the embodiment of FIG. 11, a cation exchange membrane (CEM) defines diluate and concentrate compartments in conjunction with the electrodes. From left to right in FIG. 11, the components are end plate, gasket, electrode, spacer, gasket, CEM, gasket, spacer, electrode, gasket and end plate. In some embodiments, the spacers can comprise plastic mesh in the membrane center, thereby enabling diluate and concentrate blood streams to make prolonged contact with the CEM.

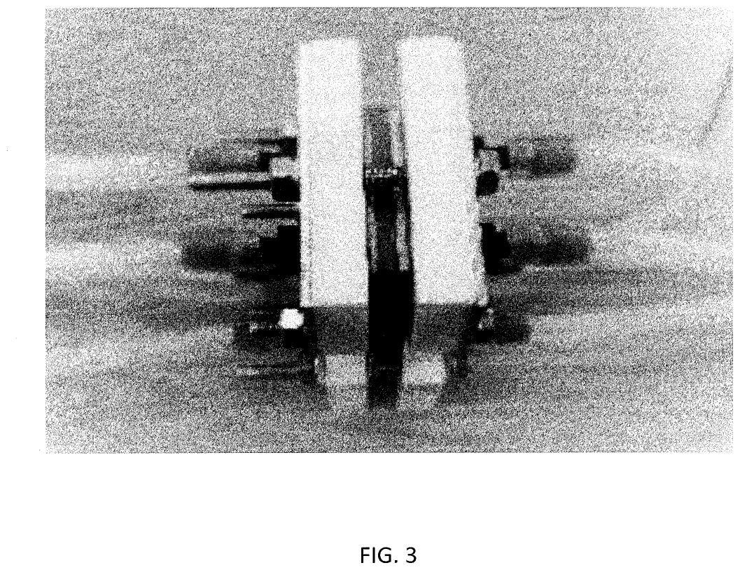

FIG. 3 is a photograph of a power source employing the membrane stack and electrodes of FIG. 1. As provided in FIG. 3, the power source comprises a first conduit for delivering the diluate blood stream to the diluate fluid compartments and a second conduit for delivering the concentrate blood stream to the concentrate fluid compartment. The power source also comprises return conduits for the diluate and concentrate blood streams.

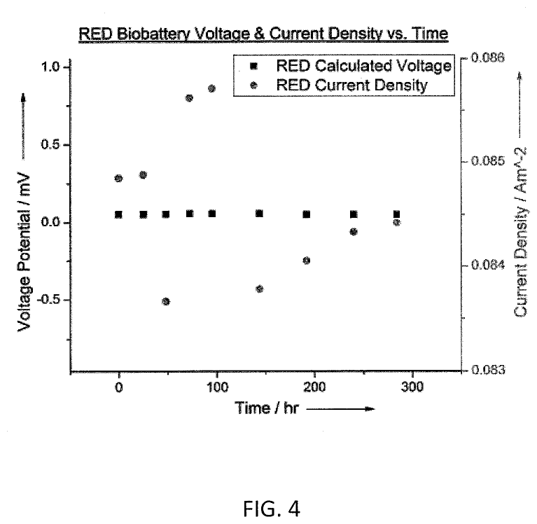

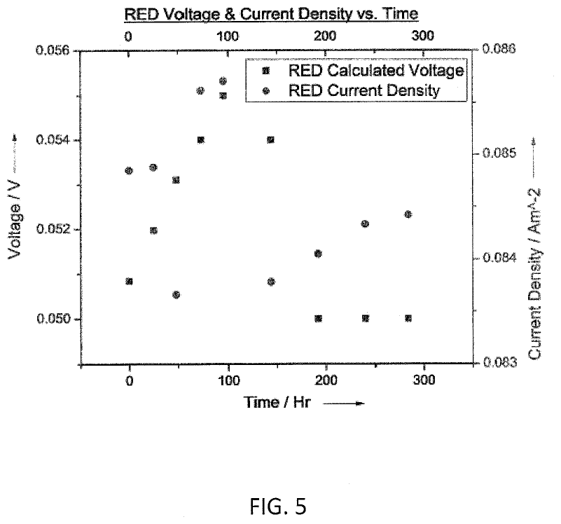

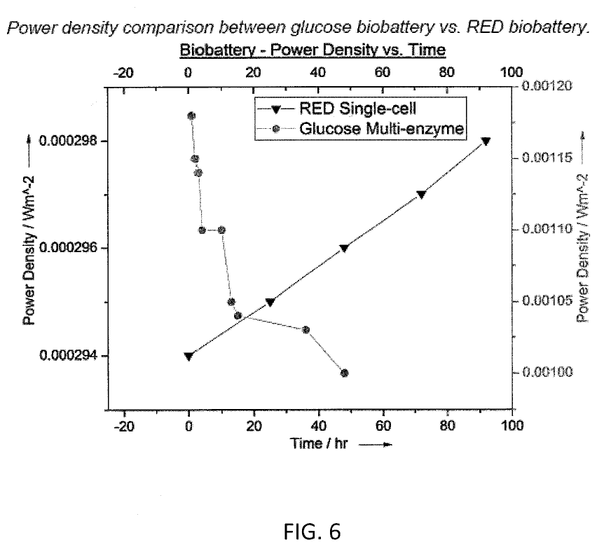

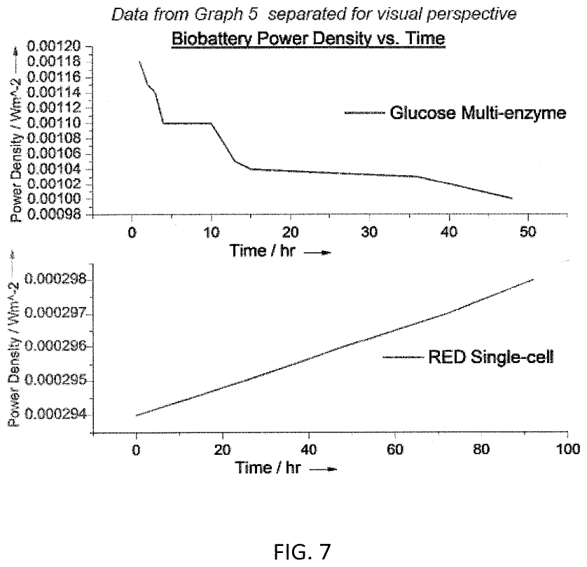

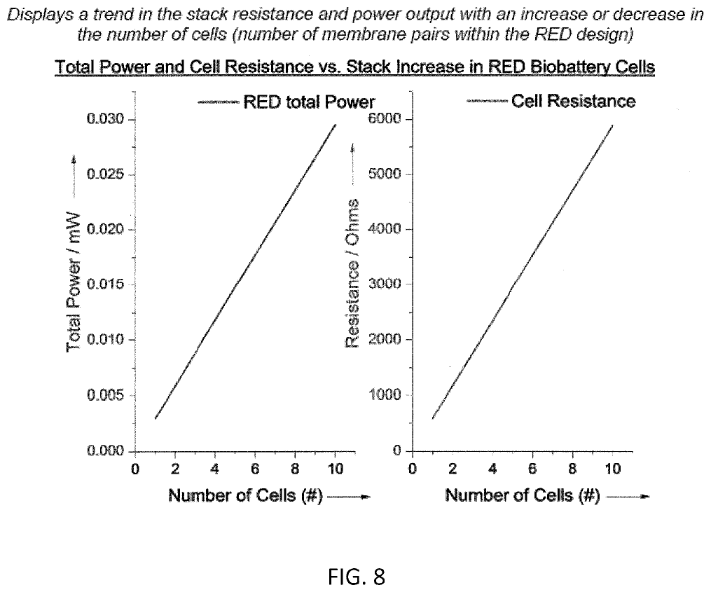

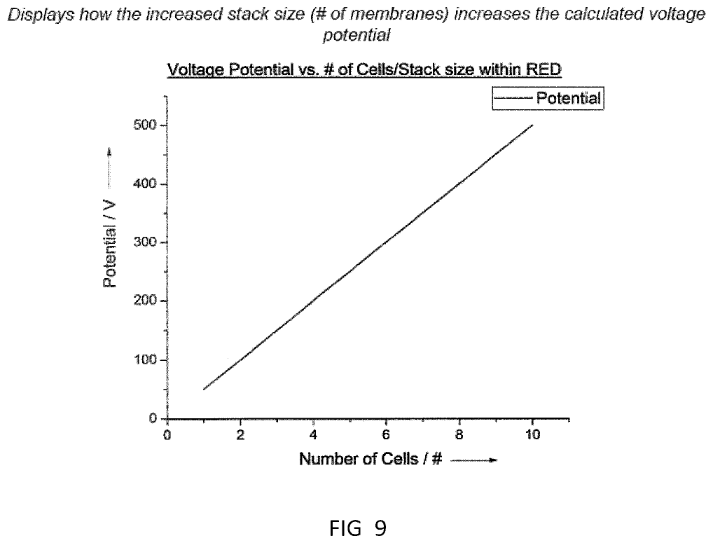

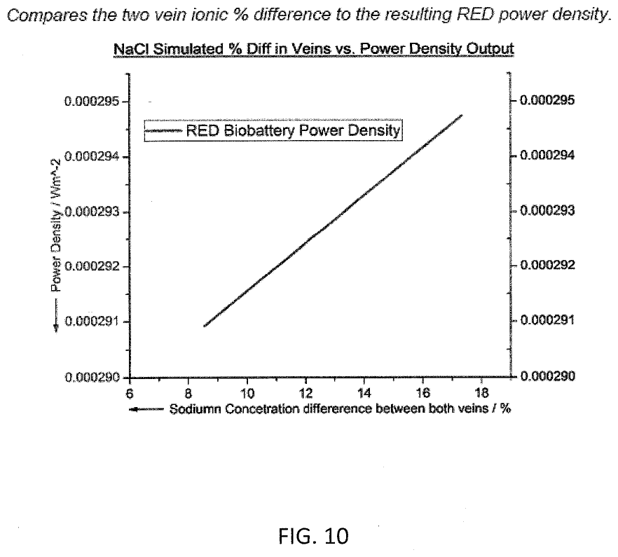

The power source pictured in FIG. 3 was employed for power generation simulations based on sodium chloride solutions mimicking diluate and concentrate blood streams. FIGS. 4 and 5 illustrate several results of the simulations. FIGS. 6 and 7 provide power density over time comparisons between a reverse electrodialysis power source described herein and a biobattery architecture based on the enzymatic breakdown of glucose. As illustrated in FIGS. 6 and 7, power density increases with time for a reverse electrodialysis power source described herein. In contrast, power density decreases significantly over time for the biobattery architecture. Power density of a reverse electrodialysis power source described herein can also be increased by increasing the number of cells in the membrane stack. FIG. 8 illustrates a linear increase in power density with increasing number of cells. Similarly, FIG. 9 illustrates a linear increase in potential with increasing number of cells. As power sources described herein operate on salinity gradients, power density also increases with increasing differences in ionic concentration between the diluate and concentrate blood streams. FIG. 10 illustrates a linear dependence of powder density relative to the magnitude of the sodium gradient between the diluate and concentrate blood streams.

In another aspect, methods of powering a medical implant are described herein. In some embodiments, a method of powering a medical implant comprises providing a power source comprising an anode and cathode adjacent to a membrane stack, the membrane stack comprising at least one ion exchange membrane defining a diluate compartment and concentrate compartment. In some embodiments, the membrane stack comprises alternating anion and cation exchange membranes defining one or more diluate and concentrate fluid compartments. A diluate blood stream is flowed into the one or more diluate compartments via a first conduit and a concentrate blood stream is flowed into the one or more concentrate compartments via a second conduit, wherein the concentrate blood stream has an ionic concentration higher than the diluate blood stream. Ions are passed through the ion exchange membrane from the concentrate blood stream, wherein the power source is connected to the medical implant for extraction of electrical current from the power source. In embodiments employing anion and cation exchange membranes, anions are passed from the concentrate blood stream through the anion exchange membrane and cations are passed from the concentrate blood stream through the cation exchange membrane, wherein the power source is connected to the medical implant for extraction of electrical current from the power source. In some embodiments, the power source is directly connected to the implant to power the implant. Alternatively, the power source can be connected to the implant via one or more intermediate electronic devices. For example, the power source can be connected to a battery, wherein the power source charges the battery for operation of the implant.

In some embodiments, the first conduit is in fluid communication with a first blood vessel, and the second conduit is in fluid communication with a second blood vessel. The first and second blood vessels, in some embodiments, are differing veins. A method described herein, in some embodiments, further comprises returning the diluate blood stream to the first vein via a first return conduit. The concentrate blood stream can be returned to the second vein via a second return conduit.

Medical implants for use with power sources described herein include any implant requiring an electrical power source. In some embodiments, the implant is a cardiac implant such as a pacemaker, implantable cardiac defibrillator or cardiac resynchronization device. In other embodiments, the implant may be a drug delivery system or bone growth generator.

In a further aspect, medical devices are described herein. A medical device, in some embodiments, comprises an implant and a power source coupled to the implant. The power source comprises an anode and cathode adjacent to a membrane stack, the membrane stack comprising at least one ion exchange membrane defining a diluate compartment and concentrate compartment. In some embodiments, the membrane stack comprises alternating anion and cation exchange membranes defining one or more diluate and concentrate fluid compartments. The power source includes a first conduit for delivering a diluate blood stream to the one or more diluate fluid compartments and a second conduit for delivering a concentrate blood stream to the one or more concentrate fluid compartments, wherein the concentrate blood stream has an ionic concentration higher than the diluate blood stream. In some embodiments, the power source is directly connected to the implant to power the implant. In other embodiments, the power source can be connected to the implant via one or more intermediate electronic devices. For example, the power source can be connected to a battery, wherein the power source charges the battery for operation of the implant.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A power source for a medical implant comprising:
an anode and cathode adjacent to a membrane stack, the membrane stack comprising at least one ion exchange membrane defining diluate and concentrate fluid compartments; and
a first conduit for delivering a diluate blood stream to the diluate fluid compartment and a second conduit for delivering a concentrate blood stream to the concentrate fluid compartment, wherein the concentrate blood stream has an ionic concentration higher than the diluate blood stream.

2. The power source of claim 1, wherein the ion exchange membrane is a cation exchange membrane.

3. The power source of claim 1, wherein the membrane stack comprises alternating anion and cation exchange membranes defining a plurality of diluate and concentrate fluid compartments.

4. The power source of claim 1 further comprising electrical circuitry for transferring electrical energy to the medical implant.

5. The power source of claim 1 further comprising a diluate blood stream return conduit and a concentrate blood stream return conduit.

6. The power source of claim 5, wherein the first conduit is in fluid communication with a first vein and the second conduit is in fluid communication with a second vein.

7. The power source of claim 1, wherein the ionic concentration is sodium ion concentration.

8. The power source of claim 7, wherein the sodium ion concentration of the concentrated blood stream is at least 10 percent higher than the sodium ion concentration of the diluate blood stream.

9. The power source of claim 7, wherein the sodium ion concentration of the concentrated blood stream is 10-30 percent higher than the sodium ion concentration of the diluate blood stream.

10. The power source of claim 1, wherein the membrane stack does not comprise one or more rinse chambers.

11. The power source of claim 3, wherein the membrane stack is formed of a single cell pair.

12. The power source of claim 3, wherein the membrane stack comprises two or more cell pairs.

13. The power source of claim 1 further comprising a salt cartridge in fluid communication with the second conduit.

14. The power source of claim 13, wherein a blood stream contacts the salt cartridge to provide the concentrate blood stream.

15. The power source of claim 1, wherein the medical implant is a cardiac implant.

16. The power source of claim 1, wherein the medical implant is an artificial organ.

17. A method of powering a medical implant comprising:
providing a power source comprising an anode and cathode adjacent to a membrane stack, the membrane stack comprising at least one ion exchange membrane defining diluate and concentrate fluid compartments;
flowing a diluate blood stream into the diluate compartment via a first conduit and flowing a concentrate blood stream into the concentrate compartment via a second conduit, wherein the concentrate blood stream has an ionic concentration higher than the diluate blood stream;
passing ions from the concentrate blood stream through the ion exchange membrane; and
connecting to the power source to the medical implant to extract electrical current from the power source.

18. The method of claim 17, wherein the ion exchange membrane is a cation exchange membrane.

19. The method of claim 17, wherein the membrane stack comprises alternating anion and cation exchange membranes defining a plurality of diluate and concentrate fluid compartments and anions are passed from the concentrate blood stream through the anion exchange membrane and cations from the concentrate blood stream are passed through the cation exchange membrane.

20. The method of claim 17, wherein the first conduit is in fluid communication with a first vein and the second conduit is in fluid communication with a second vein.

21. The method of claim 20 further comprising returning the diluate blood stream to the first vein via a first return conduit.

22. The method of claim 21 further comprising returning the concentrate blood stream to the second vein via a second return conduit.

23. The method of claim 17, wherein the ionic concentration is sodium ion concentration.

24. The method of claim 23, wherein the sodium ion concentration of the concentrated blood stream is at least 10 percent higher than the sodium ion concentration of the diluate blood stream.

25. The method of claim 23, wherein the sodium ion concentration of the concentrated blood stream is 10-30 percent higher than the sodium ion concentration of the diluate blood stream.

26. The method of claim 17, wherein the membrane stack does not comprise one or more rinse chambers.

27. The method of claim 19, wherein the membrane stack is formed of a single cell pair.

28. The method of claim 13, wherein the power source further comprises a salt cartridge in fluid communication with the second conduit.

29. The method of claim 28, wherein a blood stream contacts the salt cartridge to provide the concentrate blood stream.

30. The method of claim 17, wherein the medical implant is a cardiac implant.

31. The method of claim 17, wherein the power source is connected directly to the medical implant.

32. The method of claim 17, wherein the power source is connected to an intermediate electronic device.

33. The method of claim 32, wherein the intermediate electronic device is a rechargeable battery.

34. A medical device comprising:
an implant; and
a power source coupled the implant, the power source comprising an anode and cathode adjacent to a membrane stack, the membrane stack comprising alternating anion and cation exchange membranes defining one or more diluate and concentrate fluid compartments and a first conduit for delivering a diluate blood stream to the one or more diluate fluid compartments and a second conduit for delivering a concentrate blood stream to the one or more concentrate fluid components, wherein the concentrate blood stream has an ionic concentration higher than the diluate blood stream.
